(19) United States
(12) Patent Application Publication
(10) Pub. No.: US 2005/0085785 A1
(43) Pub. Date: Apr. 21, 2005

(54) High impact strength film and non-pvc containing container and pouch and overpouch

(76) Inventors: Sherwin Shang, Vernon Hills, IL (US); Lecon Woo, Libertyville, IL (US); Tahua Yang, Woodridge, IL (US); Michael T.K. Ling, Vernon Hills, IL (US); Craig Sandford, Buffalo Grove, IL (US)

Correspondence Address: BAXTER HEALTHCARE CORPORATION;RENAL DIVISION, 1 BAXTER PARKWAY, DF3-3E, DEERFIELD, IL, 60015, US

(21) Appl. No.: 10/688,843

(22) Filed: Oct. 17, 2003

(51) Int. Cl. A61B 19/00

(52) U.S. Cl. 604/403

(57) ABSTRACT

A monolayer film of a polymer blend of a first component selected from the group consisting of an ethylene containing polymer, the first component present in an amount by weight of the film from about 60% to about 1%, the first component having a first melting point temperature determined by DSC, a second component selected from the group consisting of propylene containing polymers and methyl pentene containing polymers, the second component being present in an amount by weight of the film from about 99% to about 40%, the second component having a second melting point temperature determined by DSC; and the film being capable of withstanding steam sterilization at a temperature from about 100° C. to about 130° C.

### CROSS-REFERENCE TO RELATED APPLICATION

Not Applicable.

### FEDERALLY SPONSORED RESEARCH OR DEVELOPMENT

Not Applicable.

### BACKGROUND OF THE INVENTION

The present invention relates generally to polymer blends for fabricating multilayer films, and more particularly to polymer blends for fabricating multilayer films that have high impact strength and optionally high barrier to water and gas transmission. The multilayer films of the present invention are suitable for fabricating primary containers and overpouches for primary containers, to be used in medical and other applications.

In the medical field, primary containers are used to collect, store, transport, and ultimately deliver therapeutic fluids, nutritional solutions, respiratory therapy agents, dialysis solutions, blood, blood products, plasma derivatives, plasma expanders, blood substitutes, anti-coagulants, blood preservatives, and other therapeutic agents. Oftentimes, these primary containers are placed into secondary containers such as an overpouch to decrease the water vapor and/or gas transmission rate to maintain the integrity and volume of the agent contained within the primary container. The primary container can be attached to a tubing set or tubing sets, and be accompanied by other containers to form a therapeutic fluid delivery set.

The overpouch must have a unique combination of properties. For example, it is desirable that the overpouch be optically transparent in order to inspect visually the contents of the primary container for contaminants to the agent contained therein. At a minimum, the transparency must permit the container's label copy to be legible.

The overpouch material must also be functional over a wide range of temperatures. For example, certain premixed drug solutions are stored and transported in containers at temperatures of −10° C. to minimize the drug degradation. Further, the same package must have the ability to withstand an autoclaving or sterilization process, which is usually accomplished using steam at temperatures of about 121° C. and at elevated pressures. Further, the overpouch material must exhibit high impact strength after exposure to such temperature abuse.

In addition, it is desirable that the overpouch provide a barrier to oxygen, moisture and carbon dioxide which may degrade the contents of the primary container.

The overpouch must also allow easy access to the inside, primary container by providing an “easy-open” feature such as a tear strip, notch, slit, or the like where no cutting implement is needed.

It is also desirable that the overpouch be free from, or have a low content of, low molecular weight additives such as plasticizers, stabilizers and the like, which could be released into the medications or biological fluids that are contained within the primary container inside the overpouch, thereby potentially causing danger to patients who are using such devices.

While these characteristics are desirable in an overpouch material, the need for an overpouch could be eliminated if these same characteristics could be achieved in the primary container itself. It is therefore desirable to produce a primary container made of a material exhibits the characteristics described above.

The present invention is provided to solve these and other problems.

### SUMMARY OF THE INVENTION

The present invention provides a monolayer film fabricated from a polymer blend of a first component selected from the group consisting of an ethylene containing polymer, the first component present in an amount by weight of the film from about 60% to about 1%, the first component having a first melting point temperature determined by DSC, a second component selected from the group consisting of propylene containing polymers and methyl pentene containing polymers, the second component being present in an amount by weight of the film from about 99% to about 40%, the second component having a second melting point temperature determined by DSC; and the film being capable of withstanding steam sterilization at a temperature from about 100° C. to about 130° C. The film after steam sterilization has sufficient impact strength to withstand a drop from 8 feet without rupturing. The film does not require that any of its components be cross-linked or that it be exposed to cross-linking radiation.

The present invention further provides a multiple layered film having a seal layer from the polymer blend described above and a second layer of a barrier material.

The present invention further provides fabricating containers for numerous purposes such as to contain medical solutions or for food products, multiple chamber containers and overpouches.

These and other aspects and attributes of the present invention will be discussed with reference to the following drawings and accompanying specification.

### BRIEF DESCRIPTION OF THE DRAWINGS

FIG. 1 is a cross-sectional view of a multiple layer film of the present invention;

FIG. 2 is a cross-sectional view of a multiple layered film of the present invention;

FIG. 3 is a flowable materials container fabricated from a film of the present invention;

FIG. 4 is a monolayer film of the present invention; and

FIG. 5 is a multiple chambered container with a peelable seal separating the chambers.

### DETAILED DESCRIPTION OF THE INVENTION:

The present invention is susceptible of embodiments in many different forms. Preferred embodiments of the invention are disclosed with the understanding that the present disclosure is to be considered as exemplifications of the principles of the invention and are not intended to limit the broad aspects of the invention to the embodiments illustrated.

The present invention provides monolayer films and multiple layer films. In a preferred form of the invention the monolayer film will be fabricated from a polymer blend containing at least two components of an ethylene containing polymer and a propylene containing polymer. The multiple layer films of the present invention include the monolayer film as a layer and have additional layers such as oxygen barrier layers from polymers or polymer blends as follows.

I. Oxygen Barrier Polymer Blends and Multilayer Films Therefrom

The polymer blends utilized in the oxygen and water barrier layer of the multilayer films described herein are ethylene vinyl alcohol copolymers and polyamide containing polymers.

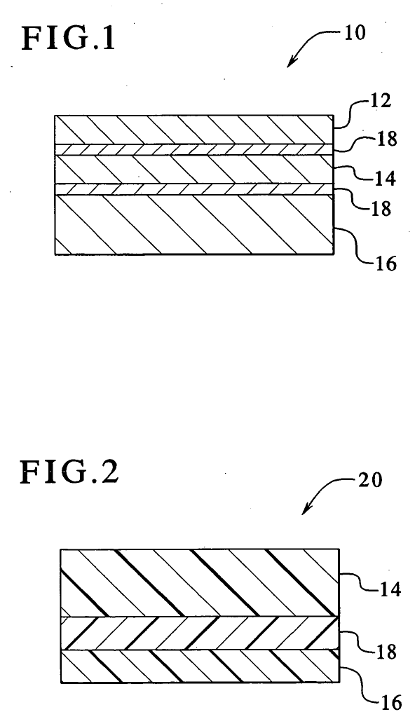

FIG. 1 shows a five layered film structure 10 having an outer layer 12, a barrier layer 14, and an inner layer 16. The film structure of FIG. 1 may also contain tie layers 18, located between the barrier layer 14 and the outer layer 12, and between the barrier layer 14 and the inner layer 16. The barrier layer 14 of the film of FIG. 1 contains an ethylene vinyl alcohol copolymer, or a polyamide containing polymer. The inner layer 16 contains a blend of an ethylene and α-olefin copolymer and a propylene containing polymer discussed in detail below. The outer layer 12, contains a material selected from the group consisting of a polyolefin, a polyamide and a polyester.

FIG. 2 shows a three layered film structure 20 having a barrier layer 14, and an inner layer 16 connected by a tie layer 18. The barrier layer 14 of this film is fabricated from a polyamide containing polymer and the inner layer 16 is the same as the inner layer of the film of FIG. 1.

A. Ethylene Vinyl Alcohol Copolymers

In one embodiment of the present invention, an ethylene vinyl alcohol (EVOH) copolymer is utilized as an oxygen barrier core layer. Suitable EVOH copolymers may be found in U.S. Pat. No. 6,083,587, incorporated herein by reference in its entirety, which discloses EVOH copolymers which have about 25% to about 45% of ethylene, and a melting point of about 150-195° C. In an embodiment, the EVOH has an ethylene content of 32 mole percent.

Other EVOH copolymers suitable for the present invention, described in U.S. Pat. No. 6,479,160, are ethylene vinyl alcohol copolymers which contain about 44% ethylene, a number average molecular weight of about 29,500 and a melting point of 164° C. Another suitable grade of EVOH copolymers has about 32% mole ethylene with a melting point of 183° C. A further suitable copolymer has about 29% ethylene, a number average molecular weight of about 22,000 and a melting point of 188° C. The EVOH layer of the invention may additionally contain a functional oxidizable polydiene or polyether as an oxygen scavenger. Suitable oxidizable polydienes are described below.

The ethylene vinyl alcohol composition may contain an ethylene vinyl alcohol copolymer grade which is retortable. The term “retort” as used herein is a process where a package is conditioned in steam at 121° C. for 30 minutes. A retortable grade of ethylene vinyl alcohol is defined as a material which remains clear without haze or microcracking after conditioning at 121° C. in steam for 30 minutes.

The oxygen barrier properties of EVOH are adversely impacted upon exposure to water. Thus, it is important to keep the EVOH barrier layer 14 of the films of the present invention dry. To this end, an outer layer 12 is used to assist in the removal of water that makes its way to the barrier layer 14 through the inner layer 16, or otherwise to maintain the oxygen barrier properties of the barrier layer 14.

The outer layer 12 used in conjunction with the EVOH barrier layer 14 may be a polyamide, polyester, polyolefin or other material that aids in the escape of water away from the barrier layer 14. Suitable polyamide and polyolefin polymer blends are discussed below. Suitable polyesters for the outer layer 12 include polycondensation products of di- or polycarboxylic acids and di or polyhydroxy alcohols or alkylene oxides. In an embodiment, the polyesters are a condensation product of ethylene glycol and a saturated carboxylic acid such as ortho or isophthalic acids and adipic acid. More preferably the polyesters include polyethyleneterphthalates produced by condensation of ethylene glycol and terephthalic acid; polybutyleneterephthalates produced by a condensations of 1,4-butanediol and terephthalic acid; and polyethyleneterephthalate copolymers and polybutyleneterephthalate copolymers which have a third component of an acid component such as phthalic acid, isophthalic acid, sebacic acid, adipic acid, azelaic acid, glutaric acid, succinic acid, oxalic acid, etc.; and a diol component such as 1,4-cyclohexanedimethanol, diethyleneglycol, propyleneglycol, etc. and blended mixtures thereof.

B. Polyamide Polymer Blends

Suitable polyamide polymer blends for use as an oxygen barrier material may be found in U.S. Pat. Nos. 5,814,384, 6,410,156, 6,423,776, and 6,479,160, which are incorporated herein by reference in their entirety. U.S. Pat. No. 6,423,776 discloses a polyamide composition prepared by combining a polyamide homopolymer, copolymer, or blends thereof, and and oxidizable polydiene or polyether. The composition may also include a metal carboxylate salt catalyst and a nanoscale clay.

In an embodiment, the polyamide homopolymer or copolymer is selected from aliphatic polyamides and aliphatic/aromatic polyamides having a molecular weight of from about 10,000 to about 100,000. Useful aliphatic polyamide homopolymers include poly(4-aminobutyric acid) (nylon 4), poly(6-aminohexanoic acid) (nylon 6, also known as poly(caprolactam)), poly(7-aminoheptanoic acid) (nylon 7), poly(8-aminooctanoic acid)(nylon 8), poly(9-aminononanoic acid) (nylon 9), poly(10-aminodecanoic acid) (nylon 10), poly(11-aminoundecanoic acid) (nylon 11), poly(12-aminododecanoic acid) (nylon 12), poly(hexamethylene adipamide) (nylon 6,6), poly(hexamethylene sebacamide) (nylon 6,10), poly(heptamethylene pimelamide) (nylon 7,7), poly(octamethylene suberamide) (nylon 8,8), poly(hexamethylene azelamide) (nylon 6,9), poly(nonamethylene azelamide) (nylon 9,9), poly(decamethylene azelamide) (nylon 10,9), poly(tetramethylene adipamide (nylon 4,6), caprolactam/hexamethylene adipamide copolymer (nylon 6,6/6), hexamethylene adipamide/caprolactam copolymer (nylon 6/6,6), trimethylene adipamide/hexamethylene azelaiamide copolymer (nylon trimethyl 6,2/6,2), hexamethylene adipamide-hexamethylene-azelaiamide caprolactam copolymer (nylon 6,6/6,9/6), poly(tetramethylenediamine-co-oxalic acid) (nylon 4,2), the polyamide of n-dodecanedioic acid and hexamethylenediamine (nylon 6,12), the polyamide of dodecamethylenediamine and n-dodecanedioic acid (nylon 12,12), as well as blends and copolymers thereof and other polyamides which are not particularly delineated here.

Of these polyamides, preferred polyamides include polycaprolactam, which is also commonly referred to as nylon 6, and polyhexamethylene adipamide, which is also commonly referred to as nylon 6,6, as well as mixtures of the same.

Exemplary of aliphatic/aromatic polyamides include poly (2,2,2-trimethyl hexamethylene terephthalamide), poly(m-xylylene adipamide) (MXD6), poly(p-xylylene adipamide), poly(hexamethylene terephthalamide) (nylon 6,T), poly(hexamethylene isophthalamide) (nylon 6,1), poly(dodecamethylene terephthalamide), polyamide 6T/61, poly(tetramethylenediamine-co-isophthalic acid) (nylon 4,I), polyamide 6/MXDT/I, polyamide MXDI, hexamethylene adipamide/hexamethylene-isophthalamide (nylon 6,6/61), hexamethylene adipamide/hexamethyleneterephthalamide (nylon 6,6/6I) and as well as others which are not particularly delineated here. Blends of two or more aliphatic/aromatic polyamides and/or aliphatic polyamides can also be used.

The polyamide component is present in the overall composition in an amount of from about 80% to about 99.9% by weight, preferably from about 90% to about 99% and more preferably from about 95% to about 98%.

The polyamide composition of the current invention also contains a functional, polyamide compatible, oxidizable polydiene or polyether as an oxygen scavenger. Such oxygen scavengers are low molecular weight, small particles which are compatible and uniformly dispersible in the polyamide.

Specific non-limiting examples of functional, oxidizable polydienes as suitable oxygen scavengers include epoxy functionalized polybutadiene (1,4 and/or 1,2), maleic anhydride grafted or copolymerized polybutadiene (1,4 and/or 1,2), epoxy functionalized polyisoprene, and maleic anhydride grafted or copolymerized polyisoprene.

Specific non-limiting examples of functional oxidizable polyethers as oxygen scavengers include amine, epoxy or anhydride functionalized polypropylene oxide, polybutylene oxide (2,3 or 1,2) and polystyrene oxide.

In an embodiment, the polyamide composition further comprises a metal fatty acid salt catalyst such as a low molecular weight metal carboxylate salt catalyst. Suitable metal fatty acid salt catalysts have a counterion which is an acetate, stearate, propionate, hexanoate, octanoate, benzoate, salicylate, and cinnamate or combination thereof. Preferably the metal fatty acid salt catalyst is a cobalt, copper or ruthenium, acetate, stearate, propionate, hexanoate, octanoate, benzoate, salicylate or cinnamate, or combinations thereof. The salt is present in the overall composition in an amount of from about 0% to about 1% by weight, preferably from about 0.001% to about 0.5% and more preferably from about 0.005% to about 0.1%. The most preferred range is from about 0.01% to about 0.05%.

C. Inner layer-Polyolefin Fluid Contact Layer

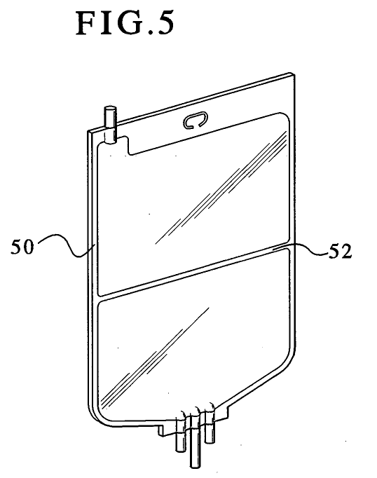

The fluid contact layer 16 of the multilayer films of the present invention shown in FIGS. 1 and 2, or as a monolayer film shown in FIG. 4, is made from a two-component blend of polyolefin polymers which are suitable for use in food applications and exhibit high impact strength when fabricated into a multilayer film.

The first component is selected from the group of: (1) ethylene and α-olefin interpolymers having a density of less than about 0.915 g/cc, (2) ethylene and lower alkyl acrylate interpolymers, (3) ethylene and lower alkyl substituted alkyl acrylate interpolymers and (4) ionic polymers, commonly referred to as ionomers. The second component is selected from the group consisting of: (1) propylene containing polymers, (2) butene containing polymers, (3) polymethyl pentene containing polymers, (4) cyclic olefin containing polymers and (5) bridged polycyclic hydrocarbon containing polymers. The film is capable of withstanding terminal steam sterilization without exposing the film to radiation or other cross-linking techniques. The first component has a first melting point temperature determined by differential scanning calorimetry (DSC) and the second component has a second melting point temperature determined by DSC that is higher than the first melting point temperature.

The film has a modulus of elasticity when measured in accordance with ASTM D882 of less than about 60,000 psi, an internal haze when measured in accordance with ASTM D1003 of less than about 25%, self adhesion ranking greater than about two (as defined below), slight or no adhesion to overpouch materials, has a sample creep at 120° C. at about 27 psi loading of less than or equal to 150%, and the film can be heat sealed into a container having seals wherein the seals remain intact when a liquid filled container is autoclaved at temperatures from about 100° C. to about 121 ° C. for one hour.

As used herein, the term “interpolymer” includes copolymers, terpolymers either random or block.

Suitable ethylene and α-olefin interpolymers preferably have a density, as measured by ASTM D-792 of less than about 0.915 g/cc and are commonly referred to as very low density polyethylene (VLDPE), ultra low density ethylene (ULDPE) and the like. The α-olefin should have from 3-17 carbons, more preferably from 4-12 and most preferably 4-8 carbons. In a preferred form of the invention, the ethylene and α-olefin copolymers are obtained using single site catalysts. Suitable single site catalyst systems, among others, are those disclosed in U.S. Pat. Nos. 5,783,638 and 5,272,236. Suitable ethylene and α-olefin copolymers include those sold by Dow Chemical Company under the AFFINITY tradename, DuPont-Dow under the ENGAGE tradename and Exxon under the EXACT and PLASTOMER tradenames. However, suitable ethylene and α-olefin copolymers can also be provided using Ziegler-Natta type catalysts.

The term “lower alkyl acrylates” refers to comonomers having the formula set forth in Diagram 1:

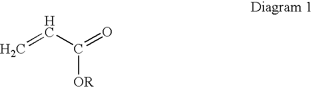

The R group refers to alkanes having from 1 to 17 carbons. Thus, the term “lower alkyl acrylates” includes but is not limited to methyl acrylate, ethyl acrylate, butyl acrylate, and the like.

The term “alkyl substituted alkyl acrylates” refers to comonomers having the formula set forth in Diagram 2:

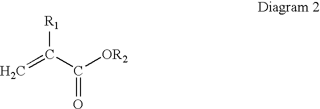

R₁ and R₂ are alkanes having 1-17 carbons and can have the same number of carbons or have a different number of carbons. Thus, the term “alkyl substituted alkyl acrylates” includes but is not limited to methyl methacrylate, ethyl methacrylate, methyl ethacrylate, ethyl ethacrylate, butyl methacrylate, butyl ethacrylate and the like.

Suitable homopolymer and copolymers of cyclic olefins and bridged polycyclic hydrocarbons and films thereof can be found in U.S. Pat. Nos. 5,218,049, 5,854,349, 5,863,986, 5,795,945, 5,792,824; and European Patent numbers EP 0 291,208, EP 0 283,164, EP 0 497,567 which are incorporated in their entirety herein by reference and made a part hereof.

In a preferred form of the invention, suitable cyclic olefin monomers are monocyclic compounds having from 5 to about 10 carbons in the ring. The cyclic olefins can be selected from the group consisting of substituted and unsubstituted cyclopentene, cyclohexene, cycloheptene, and cyclooctene. Suitable substituents include lower alkyl, acrylate derivatives and the like.

In a preferred form of the invention, suitable bridged polycyclic hydrocarbon monomers have two or more rings and more preferably contain at least 7 carbons. The rings can be substituted or unsubstituted. Suitable substitutes include lower alkyl, aryl, aralkyl, vinyl, allyloxy, (meth) acryloxy and the like. The bridged polycyclic hydrocarbons are selected from the group consisting of those disclosed in the above incorporated patents and patent applications. Suitable bridged polycyclic hydrocarbon containing polymers are sold by Ticona under the tradename TOPAS, by Nippon Zeon under the tradename ZEONEX and ZEONOR, by Daikyo Gomu Seiko under the tradename CZ resin, and by Mitsui Petrochemical Company under the tradename APEL.

In a preferred form of the present invention, a monolayered film formed from one of the above blends will have the following physical characteristics: (1) a modulus of elasticity when measured in accordance with ASTM D882 of less than about 60,000 psi, (2) an internal haze when measured in accordance with ASTM D1003 of less than about 25%, (3) self adhesion ranking greater than about two as defined below, (4) essentially no adhesion to overpouch materials, (5) has a sample creep at 120° C. at about 27 psi loading of less than or equal to 150%, and (6) the film can be heat sealed into a container having seals wherein the seals remain intact when a liquid-filled container is autoclaved at 121° C. for one hour.

The film is also sufficiently flexible to construct flowable material containers. The film has a modulus of elasticity of less than about 60,000 psi, more preferably less than about 40,000 psi, even more preferably less than about 30,000 and most preferably less than about 20,000 psi when measured in accordance with ASTM D-882. When the flowable material container is an I.V. container it is desirable the container collapse or substantially collapse upon draining, and, therefore, should have a modulus of elasticity of less than about 40,000 psi, more preferably less than about 30,000 psi, and even more preferably less than about 20,000 when measured in accordance with ASTM D-882.

For the purposes of this invention, self-adhesion is defined as the tendency of the film to adhere to itself during autoclaving. This property can be determined with the following test. Film strips are cut 8″×2″, with the larger dimension in the machine direction. These strips are rolled into 2″ long tubes approximately 0.5″ in diameter. The wound film is held in place by compressing the film layers together at one end with a paper clip. The tubes are then placed in a steam autoclave at 121° C. for 30 minutes. The samples are allowed to cool for at least one hour. The film is then unwound. The resistance to unwinding and relative damage to the film is ranked as shown in Table 1 as follows:

TABLE 1

| RANK | OBSERVED RESULT |
|---|---|
| (1) | The film cannot be unwound without destroying the film. |
| (2) | The film is difficult to peel and significant surface damage |
| | results. |
| (3) | Some resistance to peeling and minor surface damage are noted. |
| (4) | Slight resistance to peeling noted with little or no surface |
| | damage. |
| (5) | No peel resistance and no surface damage noted. |

Ranks are determined by three or more individuals and recorded as an average.

Adhesion to overpouch materials is determined by the following qualitative test. One inch wide strips of film are sealed into typical over pouch bags (medium or high density polyethylene). The over pouch bag is then placed into a laboratory autoclave at 252° F. and 24.5 psig gauge pressure for one hour. After autoclaving, the bags are cut open and the strips removed. If the films separate from the over pouch without leaving damage marks on the film surface, a ranking of no adhesion (N) is given. If the film separation produces visible damage, a ranking is given (Y) indicating that tack to the over pouch is present. A ranking to indicate slight adhesion (S) can also be given.

Creep properties were determined at 120° C. by clamping film strips having a thickness from about 5 mils to about 15 mils in a temperature controlled oven and loading with weights to produce a stress of about 27 psi. After loading for 40 minutes, the film strips were removed and the dimensional changes in a pre-marked one inch gap were recorded.

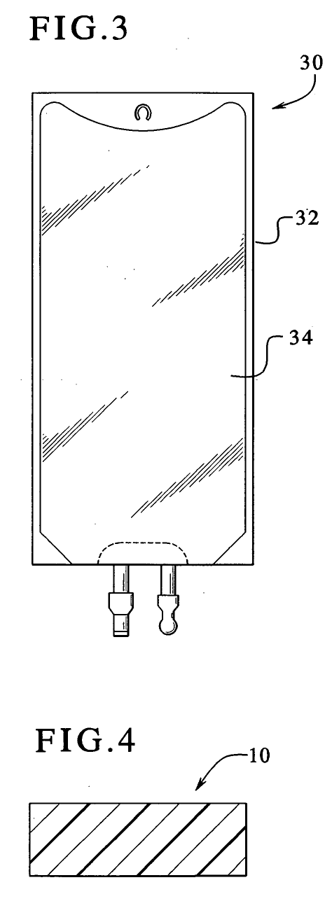

The film is capable of being sealed using standard heat sealing techniques. An adequate heat seal is formed when a fluid container, such as the one shown in FIG. 3, is fabricated from the film by sealing peripheral edges to define a centrally disposed fluid chamber. The container is filled with water and subjected to a standard autoclave sterilization process. Adequate heat seals remain intact upon completion of the autoclave cycle.

The films of the present invention have a haze of less than about 25% and most preferably less than about 15% when measured in accordance with ASTM D1003. For the purposes of this invention, internal haze is defined as the haze value measured when both film surfaces have been wetted with isopropyl alcohol.

The first component is present in an amount by weight of the polymer blend from 1% to about 60%, more preferably from 5%-60% and more preferably from about 10% to about 50%

The first component can be a single ethylene-containing polymer or a blend of two or more ethylene containing polymers which in sum constitute by weight the ranges set forth for the first component. The melting point temperature of such a blend will show a single distinct composite melting point or a peak for each ethylene-containing polymer of the blend or a combination of the same.

Suitable ethylene-containing polymers include those selected from the group consisting of ethylene homopolymers and ethylene copolymers set forth above. Suitable ethylene and α-olefin copolymers will have a density of less than about 0.915 g/cc, more preferably less than about 0.905 g/cc, and most preferably less than about 0.900 g/cc. Suitable polymers include, but are not limited to, ultra low-density polyethylene (ULDPE), ethylene-propylene rubber (EPR), and ethylene propylene diene terpolymer (EPDM). Preferably, the ethylene-containing polymers are those sold by Dow Chemical Company under the AFFINITY tradename, most preferably Affinity PL 1880 and VP 8770, and by DuPont-Dow under the ENGAGE tradename, most preferably Engage 8003.

The second component will constitute the remaining weight-percent portion of the blends and will be present singularly or in sum the converse weight percentage ranges from those set forth above for the first component. Accordingly, if the first component is present from about 99% to about 40%, the second component or the sum of the additional components will be the converse or from about 5% to about 50%.

The second component may be a single propylene-containing polymer or a single methyl-pentene-containing polymer. The second component can also be a blend of two or more propylene-containing polymers, two or more methyl-pentene-containing polymers or a blend of at least one propylene-containing polymer and at least one methyl-pentene-containing polymer.

Suitable propylene-containing polymers include those selected from the group consisting of homopolymers of polypropylene, copolymers and terpolymers of propylene with one or more comonomers selected from α-olefin having from 2-18 carbons. Suitable polypropylene copolymers and terpolymers include random or block propylene and ethylene copolymers or random or block propylene/ethylene/butene terpolymers. Suitable propylene and α-olefin copolymers are sold by Basell under the tradename PRO FAX, preferably PRO FAX SA-861 and by Exxon as Exxon PP3505GE1. In a preferred form of the invention, the second component will have a distinct melting point temperature, a distinct composite melting point temperature or a melting point temperature associated with each of the sub-components of the second component, or a combination of the same, determined by DSC of equal to or higher than about 135° C. Further, in a preferred form of the invention the first component will have a modulus of elasticity of less than about 200,000 psi, more preferably less than about 150,000 psi, and most preferably less than about 100,000 psi.

It may also be desirable to use a high melt strength polypropylene. High melt strength polypropylenes can be a homopolymer or copolymer of polypropylene having a melt flow index within the range of 10 grams/10 min. to 800 grams/10 min., more preferably 30 grams/10 min. to 200 grams/10 min, or any range or combination of ranges therein. High melt strength polypropylenes are known to have free-end long chain branches of propylene units. Methods of preparing polypropylenes which exhibit a high melt strength characteristic have been described in U.S. Pat. Nos. 4,916,198; 5,047,485; and 5,605,936 which are incorporated herein by reference and made a part hereof. One such method includes irradiating a linear propylene polymer in an environment in which the active oxygen concentration is about 15% by volume with high energy ionization energy radiation at a dose of 1 to 10⁴ megarads per minute for a period of time sufficient for a substantial amount of chain scission of the linear propylene polymer to occur but insufficient to cause the material to become gelatinous. The irradiation results in chain scission. The subsequent recombination of chain fragments results in the formation of new chains, as well as joining chain fragments to chains to form branches. This further results in the desired free-end long chain branched, high molecular weight, non-linear, propylene polymer material. Radiation is maintained until a significant amount of long chain branches form. The material is then treated to deactivate substantially all the free radicals present in the irradiated material.

High melt strength polypropylenes can also be obtained as described in U.S. Pat. No. 5,416,169, which is incorporated in its entirety herein by reference and made a part hereof, when a specified organic peroxide (di-2-ethylhexyl peroxydicarbonate) is reacted with a polypropylene under specified conditions, followed by melt-kneading. Such polypropylenes are linear, crystalline polypropylenes having a branching coefficient of substantially 1, and, therefore, has no free end long-chain branching and will have a intrinsic viscosity of from about 2.5 dl/g to 10 dl/g.

The present invention further contemplates utilizing polypropylene polymers obtained in a process using Ziegler-Natta and more preferably single-site and metallocene catalysts.

The present invention also contemplates using blends of propylene containing polymers as the second component of the film. In a preferred form of the invention the blends include at least a first propylene containing polymer and a second propylene containing polymer. The first propylene containing polymer and the second propylene containing polymer can be selected from the propylene homopolymer, copolymers and terpolymers set forth above. In a preferred form of the invention the first propylene containing polymer differs from the second propylene containing polymer in at least one of two ways. The first difference is the first propylene containing polymer preferably should have a melt flow rate of from about 3 times greater and more preferably from about 5 times greater than the melt flow rate of the second propylene containing polymer. The second difference is the first propylene-containing polymer preferably has a melting point from at least about 5° C. higher and more preferably from at least about 10° C. higher than that of the second propylene containing polymer. The melting point is measured in accordance with ASTM D3417 (Enthalpies of Fusion and Crystallization of Polymers by Differential Scanning Calorimetry). The first propylene containing polymer can differ from the second propylene containing polymer by the first difference, by the second difference or by both.

Suitable methylpentene-containing polymers include homopolymers of 4-methylpentene-1; copolymers and terpolymers of methylpentene with one or more comonomers selected from α-olefins having from 2-18 carbons. A preferred methylpentene-containing polymer is sold by Mitsui Petrochemical, Ltd. under the tradename TPXJ.

In a preferred form of the invention, the first component will constitute what is known as the continuous phase and the second component or other additional components will constitute a dispersed phase or dispersed phases as the case may be.

It is also contemplated additional polymer processing components can be added to the blends of the present invention. For example, it may be desirable to add a fatty acid amide or diatomaceous earth. Suitable fatty amides include those derived from fatty acids having from 10 to 30 carbons and most preferably is derived from erucic acid.

The second polymer blend can be fabricated into a monolayer film using standard polymer processing techniques such as extrusion.

As used herein, the term “interpolymer” includes copolymers, terpolymers either random or block.

D. Tie Layers

Suitable tie layers, described in U.S. Pat. No. 6,083,587, include modified polyolefins blended with unmodified polyolefins. The modified polyolefins are typically polyethylene or polyethylene copolymers. The polyethylenes can be ultra low density polyethylene (ULDPE), low density polyethylene (LDPE), linear low density polyethylene (LLDPE), medium density polyethylene (MDPE), and high density polyethylenes (HDPE). The modified polyethylenes may have a density from 0.850-0.95 g/cc.

The polyethylene may be modified by grafting with carboxylic acids, and carboxylic anhydrides. Suitable grafting monomers include, for example, maleic acid, fumaric acid, itaconic acid, citraconic acid, allylsuccinic acid, cyclohex-4-ene-1,2-dicarboxylic acid, 4-methylcyclohex-4-ene-1,2-dicarboxylic acid, bicyclo[2.2.1]hept-5-ene-2,3-dicarboxylic acid, x-methylbicyclo[2.2.1]hept-5-ene-2,3-dicarboxylic acid, maleic anhydride, itaconic anhydride, citraconic anhyride, allylsuccinic anhydride, citraconic anhydride, allylsuccinic anhydride, cyclohex-4-ene- 1,2-dicarboxylic anhydride, 4-methylcyclohex-4-ene-1,2-dicarboxylic anhydride, bicyclo[2.2.1]hept-5-ene2,3-dicarboxylic anhydride, and x-methylbicyclo[2.2. 1 ]hept-5-ene-2,2-dicarboxylic anhydride.

Examples of other grafting monomers include C₁-C₈ alkyl esters or glycidyl ester derivatives of unsaturated carboxylic acids such as methyl acrylate, methyl methacrylate, ethyl acrylate, ethyl methacrylate, butyl acrylate, butyl methacrylate, glycidyl acrylate, glycidal methacrylate, monoethyl maleate, diethyl maleate, monomethyl maleate, diethyl maleate, monomethyl fumarate, dimethyl fumarate, monomethyl itaconate, and diethylitaconate; amide derivatives of unsaturated carboxylic acids such as acrylamide, methacrylamide, maleicmonoamide, maleic diamide, maleic N-monoethylamide, maleic N,N-dietylamide, maleic N-monobutylamide, maleic N,N dibutylamide, fumaric monoamide, fumaric diamide, fumaric N-monoethylamide, fumaric N,N-diethylamide, fumaric N-monobutylamide and fumaric N,N-dibutylamide; imide derivatives of unsaturated carboxylic acids such as maleimide, N-butymaleimide and N-phenylmaleimide; and metal salts of unsaturated carboxylic acids such as sodium acrylate, sodium methacrylate, potassium acrylate and potassium methacrylate. More preferably, the polyolefin is modified by a fused ring carboxylic anhydride and most preferably a maleic anhydride.

The unmodified polyolefins can be selected from the group consisting of ULDPE, LLDPE, MDPE, HDPE and polyethylene copolymers with vinyl acetate and acrylic acid. Suitable modified polyolefin blends are sold, for example, by DuPont under the tradename BYNEL, by Chemplex Company under the tradename PLEXAR, and by Quantum Chemical Co. under the tradename PREXAR.

### II. Polymer and Film Processing

The above polymer blends may be processed into a layered structure by standard techniques well known to those of ordinary skill in the art. One processing method which may be employed is a blown film extrusion and water quench process wherein the films are extruded at high temperatures. Another processing method which may be employed is a blown film extrusion and air quench process wherein the films are extruded at high temperatures. In this process, the inner tube is sterilized from the high temperature extrusion, and filtered clean or sterilized air is used to inflate the tube. Sections of the tube are then sealed to form containers for food applications. The air in the tube is expelled before the containers are sealed on both sides. There is no contamination to the inner portion of the container prior to production since both sides of tube are sealed. This ensures that sterilized food product will not be contaminated by the containers of the present invention, which will additionally make the containers of the present invention suitable for aseptic packaging applications.

The films may also be prepared using, for example, extrusion, coextrusion, extrusion coating, blown film extrusion, cast extrusion, calendaring, lamination, blow molding or other processes that are well known in the art.

### III. Fluid Containers

The multilayer films described above are suitable for fabricating large food containers to be used in retort heating applications. Food containers made with the films of the present invention maintain high impact strength after heat treatment, and are able to further withstand subsequent freezing and microwave heating without failure.

FIG. 3 shows a container 30 made with the multilayer films of FIGS. 1, 2 or monolayer film of FIG. 4. The container 30 is heat sealed along a peripheral edge 32 of the container to define a centrally disposed chamber 34 for fluid, or semi-solid food products. These films may also be used to fabricate a multiple chamber container (FIG. 5) having a peelable seal 52 separating the chambers and a permanent seal 50 about the periphery. Such peel seal containers are disclosed in U.S. Patent Application Publication No. 2002/0115795 which is incorporated herein in its entirety by reference.

The polyolefin fluid contact layer 16 of the present invention is capable of being heat sealed using standard heat sealing techniques. An adequate heat seal is formed when a container, such as the one shown in FIG. 3, is fabricated from the film by sealing peripheral edges to define a centrally disposed fluid chamber. The container is filled with water and subjected to a standard retort sterilization process. Adequate heat seals remain intact upon completion of the retort cycle. The films of the present invention can be fabricatred into a container and filled by form, fill and seal processes that are well known in the art.

The polyolefin fluid contact layer described above imparts significant impact resistance to containers made with the multilayer films of the present invention. The multilayer films of the current invention when made into sealed pouches holding up to 6.5 liters are able to withstand an impact strength of 8 feet without failure.

### IV. Physical Properties

A. Impact Strength

One of the problems associated with institutional-sized plastic containers for packaging liquid or semi-liquid materials is their inability to withstand impact forces following heat sterilization. The heat weakens the seals and the hydraulic forces on dropping creates high stresses in the horizontal direction. Thus, an essential property of each pouch or container is that it is capable of withstanding impacts during handling and transport. The industry has developed a number of tests for determining the suitability of the pouches and containers for shipping in cases; two of which are ASTM and NSTA standards. In addition, it has been found that drop tests on individual pouches provides a reliable test for qualitatively determining the impact strength of pouches. These tests involve multiple drops of each pouch before retorting.

The containers made with the polymer films of the present invention can withstand a impact strength from 8 feet for a 6 liter container without failure.

B. High and Low Temperature Performance

The flowable materials container of the present invention can be subjected to −20° C. conditions without becoming too brittle and can be heated, such as through the use of a microwave oven, to 100° C. The container can also be subjected to an autoclave sterilization process where the film or container of the present invention is subjected to 121° C. for one hour.

C. Film Thickness

The relative thickness of the layers of the structure 10 is for example as follows for a 6.5 L volume container: the core layer should have a thickness from 3.5 mil, to about 5.5 mil or any range or combination of ranges therein. The outer layer preferably has a thickness from about 0.5 to about 2.5 mil, or any range or combination of ranges therein. The inner layer 16 has a thickness from about 0.5 to about 2.5 mil or any range or combination of ranges therein.

D. Film and Container Sterilization

The films and containers herein are capable of being sterilized using numerous techniques including terminal steam sterilization, exposure to radiation, and exposure to ethylene oxide. Terminal sterilization typically includes exposing the film or container to steam at a temperature higher than 100° C., typically 121° C., but also includes up to 130° C. Typically, terminal sterilization utilizes steam at 121° C. for time periods of from 30 minutes to 1 hour.

While specific embodiments have been illustrated and described, numerous modifications come to mind without departing from the spirit of the invention and the scope of protection is only limited by the scope of the accompanying claims.

1. A monolayer film comprising:
a polymer blend of a first component selected from the group consisting of an ethylene containing polymer, the first component present in an amount by weight of the film from about 60% to about 1%, the first component having a first melting point temperature determined by DSC, a second component selected from the group consisting of propylene containing polymers and methyl pentene containing polymers, the second component being present in an amount by weight of the film from about 99% to about 40%, the second component having a second melting point temperature determined by DSC; and the film being capable of withstanding steam sterilization at a temperature from about 100° C. to about 130° C.

2. The film of claim 1, wherein the second melting point temperature is higher than the first melting point temperature.

3. The film of claim 1 wherein the ethylene containing polymer is obtained using a catalyst selected from the group consisting of: Ziegler-Natta and single-site.

4. The film of claim 1, wherein the ethylene containing polymer is selected from the group consisting of: ethylene homopolymers, and ethylene copolymers.

5. The film of claim 1, wherein the propylene containing polymer is selected from the group of propylene homopolymers and propylene copolymers.

6. The film of claim 5, wherein the propylene containing polymer is obtained using a catalyst selected from the group consisting of: Ziegler-Natta and single-site.

7. The film of claim 1, wherein the propylene containing polymer is a high melt strength polymer.

8. The film of claim 7, wherein the high melt strength propylene containing polymer is selected from the propylene containing propylene made from electron beam process and reactor made process.

9. The film of claim 1 further comprising an oxygen scavenger.

10. The film of claim 9, wherein the oxygen scavenger is an oxidizable polydiene.

11. The film of claim 9, wherein the oxygen scavenger is an oxidizable polyether.

12. The film of claim 1, wherein the film is prepared by a process selected from the group consisting of extrusion, canlendering, blown film extrusion and blown molding.

13. The film of claim 1, is capable of being fabricated into a liquid filled container wherein the container has sufficient impact strength to resist rupturing when dropped from 8 feet.

14. The film of claim 1, is capable of being fabricated into a liquid filled container wherein the container is capable of being terminally sterilized by exposure to steam at 121° C. for one hour.

15. The film of claim 1 is capable of being sterilized by steam sterilization exposure to radiation and exposure to ethylene oxide.

16. The film of claim 1 is capable of forming a peel seal to form a multiple chambered container.

17. The film of claim 16 is further capable of forming a permanent seal to form a multiple chambered container

18. A multilayer film comprising:
a barrier layer;
a seal layer comprising a blend of: (i) an ethylene and α-olefin copolymer having a density of less than about 0.915 g/cc, and in an amount of from about 60% to about 1% by weight of the blend, and (ii) a propylene containing polymer in an amount by weight of the blend from about 99% to about 40%; and
the film can be heat sealed into a container having seals wherein the seals remain intact when the container is retorted at 121° C. for sixty minutes, and wherein the container does not rupture when dropped from 8 feet.

19. The film of claim 18, wherein the barrier layer contains a barrier material selected from the group consisting of polyamides and ethylene vinyl alcohol copolymers.

20. The film of claim 18, wherein the propylene containing polymer is selected from the group of propylene homopolymers and propylene copolymers.

21. The film of claim 20, wherein the propylene containing polymer is obtained using a catalyst selected from the group consisting of: Ziegler-Natta and single-site.

22. The film of claim 20, wherein the propylene contain polymer is a high melt strength polymer.

23. The film of claim 18, wherein the propylene containing polymer is a blend of a first propylene containing polymer and a second propylene containing polymer.

24. The film of claim 18, wherein the first propylene containing polymer has a first melt flow rate and the second propylene containing polymer has a second melt flow rate wherein the first melt flow rate is higher than the first melt flow rate.

25. The film of claim 24, wherein the first melt flow rate is about 3 times greater than the second melt flow rate.

26. The film of claim 24, wherein the first melt flow rate is about 5 times greater than the second melt flow rate.

27. The film of claim 23, wherein the first propylene containing polymer has a first melting point temperature and the second propylene containing polymer has a second melting point temperature wherein the first melting point temperature is higher than the second melting point temperature by at least about 5° C.

28. The film of claim 23, wherein the first propylene containing polymer has a first melting point temperature and the second propylene containing polymer has a second melting point temperature wherein the first melting point temperature is higher than the second melting point temperature by at least about 10° C.

29. The film of claim 1, wherein the seal layer blend further comprises an oxygen scavenger.

30. The film of claim 19, wherein the barrier layer blend further comprises an oxygen scavenger.

31. The film of claim 29, wherein the oxygen scavenger is an oxidizable polydiene.

32. The film of claim 29, wherein the oxygen scavenger is an oxidizable polyether.

33. The film of claim 18, wherein the barrier layer further comprises a metal fatty acid salt.

34. The film of claim 18, wherein the α-olefin has from 3 to 17 carbons.

35. The film of claim 18, wherein the α-olefin has from 4 to 8 carbons.

36. The film of claim 18, wherein the ethylene and α-olefin copolymer is obtained using a single site catalyst.

37. The film of claim 18, wherein the film further comprises a tie layer adhering the barrier layer to the inner layer.

38. The film of claim 18, is capable of being terminally sterilized by other sterilization modes in radiation and ethylene oxide.

39. The film of claim 18, is capable of forming a peel seal to form a multiple chambered container.

40. The film of claim 18, is capable of forming a permanent seal to form a multiple chambered container

41. A container comprising:
a wall defining a chamber, the wall having a barrier layer;
a seal layer of a polymer blend of a first component selected from the group consisting of an ethylene containing polymer, the first component present in an amount by weight of the film from about 60% to about 1%, the first component having a first melting point temperature determined by DSC, a second component selected from the group consisting of propylene containing polymers and methyl pentene containing polymers, the second component being present in an amount by weight of the film from about 99% to about 40%, the second component having a second melting point temperature determined by DSC; and the container being capable of withstanding steam sterilization at a temperature at 121° C. for sixty minutes and has an impact strength sufficient to withstand a drop from a height of 8 feet without rupturing

42. The container of claim 41, wherein the propylene containing polymer is selected from the group of propylene homopolymers and propylene copolymers.

43. The container of claim 42, wherein the propylene containing polymer is obtained using a catalyst selected from the group consisting of: Ziegler-Natta and single-site.

44. The container of claim 41, wherein the propylene contain polymer is a high melt strength polymer.

45. The container of claim 41, wherein the propylene containing polymer is a blend of a first propylene containing polymer and a second propylene containing polymer.

46. The container of claim 45, wherein the first propylene containing polymer has a first melt flow rate and the second propylene containing polymer has a second melt flow rate wherein the first melt flow rate is higher than the first melt flow rate.

47. The container of claim 45, wherein the first melt flow rate is about 3 times greater than the second melt flow rate.

48. The container of claim 45, wherein the first melt flow rate is about 5 times greater than the second melt flow rate.

49. The container of claim 45, wherein the first propylene containing polymer has a first melting point temperature and the second propylene containing polymer has a second melting point temperature wherein the first melting point temperature is higher than the second melting point temperature by at least about 5° C.

50. The container of claim 45, wherein the first propylene containing polymer has a first melting point temperature and the second propylene containing polymer has a second melting point temperature wherein the first melting point temperature is higher than the second melting point temperature by at least about 10° C.

51. The container of claim 41, wherein the seal layer blend further comprises an oxygen scavenger.

52. The container of claim 41, wherein the barrier layer blend further comprises an oxygen scavenger.

53. The container of claim 52, wherein the oxygen scavenger is an oxidizable polydiene.

54. The container of claim 52, wherein the oxygen scavenger is an oxidizable polyether.

55. The container of claim 41, wherein the barrier layer further comprises a metal fatty acid salt.

56. The container of claim 41, wherein the ethylene-containing polymer is an α-olefin.

57. The container of claim 56, wherein the α-olefin has from 3 to 17 carbons.

58. The container of claim 41, wherein the α-olefin has from 4 to 8 carbons.

59. The container of claim 41, wherein the ethylene and α-olefin copolymer is obtained using a single site catalyst.

60. The container of claim 41, wherein the container further comprises a tie layer adhering the barrier layer to the inner layer.

61. The container of claim 41, wherein the barrier layer is selected from ethylene vinyl alcohol and polyamide.

62. The container of claim 41, wherein the container has peel seal to form multiple chambers.

63. The container of claim 41, wherein the container stores fluid at −20° C. and microwave oven heating at 100° C.

64. The container of claim 41, wherein the container stores fluid at −20° C. and autoclave at 121° C.

65. The container of claim 41, wherein the container is made from an aseptic tube film manufacturing method by using 0.22 μm air filter and filtered water quench during blown film process for empty bags suitable for aseptic filling and high end fluid bags for hot fluid filling.

66. The container of claim 41, wherein the container surface is non-sticky after the heating at 121° C.
